(19) United States
(12) Patent Application Publication
(10) Pub. No.: US 2024/0331881 A1
(43) Pub. Date: Oct. 3, 2024

(54) PATIENT EXPERIENCE UPDATES BASED ON ONGOING PATIENT AND CAREGIVER METRICS

(71) Applicant: Hill-Rom Services, Inc., Batesville, IN (US)

(72) Inventors: Teresa Brasac, Miami Shores, FL (US); Patrick Douglas Harrison, Fort Lauderdale, FL (US); Eva Monheit, New York City, NY (US); Daniel Chase Shirley, Raleigh, NC (US); Autumn Thompson, Philadelphia, PA (US); Kate Vignoe, Peapack, NJ (US); Ann Wallinger, Mt. Jackson, VA (US)

(21) Appl. No.: 18/620,556

(22) Filed: Mar. 28, 2024

Related U.S. Application Data
(60) Provisional application No. 63/493,702, filed on Mar. 31, 2023.

(51) Int. Cl. G16H 80/00

(52) U.S. Cl. CPC G16H 80/00

(57) ABSTRACT

A system is provided that monitors information from locating equipment and nurse call equipment and that patient satisfaction and caregiver assignments can be optimized based on a caregiver load score and a patient care experience score.

### CROSS-REFERENCE TO RELATED APPLICATIONS

This application claims priority under 35 U.S.C. § 119 (e) to U.S. Provisional Application No. 63/493,702, filed Mar. 31, 2023, which is expressly incorporated by reference herein.

### BACKGROUND

The present disclosure relates to healthcare information systems and particularly, to healthcare information systems having nurse call systems networked with locating systems. More particularly, the present disclosure relates to a real-time patient experience monitoring system of the healthcare information system.

After a patient's stay at a healthcare facility, the patient oftentimes fills out a patient satisfaction survey. For example, the Hospital Consumer Assessment of Healthcare Providers and Systems (HCAHPS) survey has been in use since about 2006 for such purposes. The HCAHPS survey created a national standard throughout the United States for collecting and reporting patient satisfaction information that enables comparisons to be made across participating hospitals to support consumer choice. However, determining patient satisfaction based on these surveys does not provide a real-time assessment of patient experience. Furthermore, such surveys only provide information about staffing load after the fact. Accordingly, hospitals would welcome a system that informs hospital staff, such as caregivers, factors that impact patient experience while the patient is still in the hospital so that corrective measures can be taken while the patient is in the hospital to improve treatment. Such step can include assessing staff assignments and appropriately determining new patient assignments.

### SUMMARY

An apparatus, system, or method may comprise one or more of the features recited in the appended claims and/or the following features which, alone or in any combination, may comprise patentable subject matter:

According to a first aspect of the present disclosure, system for providing real-time patient experience feedback to a caregiver includes locating equipment, nurse call equipment and a patient experience module. The locating equipment tracks the location of caregivers in a healthcare facility. The nurse call equipment receives nurse call requests from patients located in patient rooms of the healthcare facility. The nurse call equipment includes at least one nurse call computer that tracks caregiver response time for nurse call requests, the time at least one caregiver is present in a patient room based on information received by the nurse call computer from the locating equipment, and how often a patient calls the caregivers, and the status of rounds that the caregivers undertake. The patient experience module receives information from the nurse call equipment, the locating equipment, and information about staffing assignments, updates a patient experience counter associated with a particular patient based on the information received on an hourly basis, and issues a notification to a caregiver of the change in patient experience counter.

In some embodiments of the first aspect, the patient experience module increases the patient experience counter for a patient if caregivers have spent at least a threshold amount of time with the patient in the previous 12 hours. In other embodiments of the first aspect, the threshold amount of time is 80 minutes.

In some embodiments of the first aspect, the patient experience module increases the patient experience counter for a patient if the patient has called the caregivers at least a threshold amount of hours with the patient in the previous 24 hours. In other embodiments of the first aspect, the threshold amount of hours is 7 hours.

In some embodiments of the first aspect, the patient experience module increases the patient experience counter for a patient if a caregiver response time to each patient call from the patient in the previous 6 hours has been more than a threshold response time.

In some embodiments of the first aspect, the threshold response time is 5 minutes.

In some embodiments of the first aspect, the patient experience module increases the patient experience counter for a patient for every partial, missed, or on-hold round in the previous 6 hours.

In some embodiments of the first aspect, the nurse call equipment includes nurse call input devices including handheld pillow speaker units having a plurality of nurse call buttons, each nurse call button corresponding to a call type that is different from each of the other nurse call buttons.

In some embodiments of the first aspect, the nurse call input devices include a plurality of nurse call buttons provided on hospital beds located in patient rooms, each nurse call button corresponding to a call type that is different from each of the other nurse call buttons.

In some embodiments of the first aspect, the notification further comprises at least one of the following: (1) a message appearing on a wireless communication device carried by at least one caregiver or (2) a message appearing on a status board display located in a caregiver work area.

According to a second aspect of the present disclosure, system for providing real-time patient experience feedback to a caregiver includes locating equipment, nurse call equipment and a patient experience module. The locating equipment tracks the location of caregivers in a healthcare facility. The nurse call equipment receives nurse call requests from patients located in patient rooms of the healthcare facility and includes at least one nurse call computer that tracks caregiver response time to each nurse call request. The nurse call computer tracks how long at least one caregiver is present in each patient room based on information received by the nurse call computer from the locating equipment. The nurse call computer tracks how often each patient calls the caregivers, and status of each round outcome that the caregivers undertake. The patient experience module receives information from the nurse call equipment, the locating equipment, and information about staffing assignments, updates a staff load counter associated with a plurality of patients based on the information, divides the plurality of patients into groups based, and assigns a new unassigned patient to a group with the lowest total staff load counter value.

In some embodiments of the second aspect, the patient experience module increases the staff load counter associated with a patient if the total number of patient calls for the patient is greater than an average benchmark for patient calls.

In some embodiments of the second aspect, the patient experience module increases the staff load counter for a patient if a total number of equipment calls for the patient is greater than an average benchmark for equipment calls.

In some embodiments of the second aspect, the patient experience module increases the staff load counter for a patient if a total number of unpredictable calls for the patient is greater than an average benchmark for unpredictable calls.

In some embodiments of the second aspect, the patient experience module increases the staff load counter for a patient if total patient visit length is greater than an average benchmark patient visit length for patient visit.

In some embodiments of the second aspect, the patient experience module increases the staff load counter for a patient if a total number of patient visits is greater than an average number of patient visits for the plurality of patients.

In some embodiments of the second aspect, the patient experience module is creates the groups by computing a descending worst fit algorithm, ranks the plurality of patients with a highest staff load counter to a lowest staff load counter, and further divides the plurality of patients into groups based on an input parameter.

In some embodiments of the second aspect, the patient experience module is further assigns the new patient to a group that minimizes bedside shift handoffs if there is a tie for the group with the lowest total staff load score.

In some embodiments of the second aspect, the information regarding types of nurse calls is used to calculate the staff load counter.

In some embodiments of the second aspect, the types of nurse calls comprises patient calls, equipment calls, and unpredictable calls.

In some embodiments of the second aspect, the patient experience module provides a notification comprising staff assignments as at least one of the following: (1) a message appearing on a wireless communication device carried by at least one caregiver or (2) a message appearing on a status board display located in a caregiver work area or (3) a message appearing on a master nurse station computer of the nurse call system.

According to a third aspect of the present disclosure, a system for providing real-time patient experience feedback to a caregiver includes locating equipment, nurse call equipment, and an electronic medical records system. The locating equipment tracks the location caregivers in a healthcare facility. The nurse call equipment receives nurse call requests from patients located in patient rooms of the healthcare facility, the patient rooms being divided up into at least a first unit of the healthcare facility and a second unit of the healthcare facility. The nurse call equipment includes at least one nurse call computer that tracks of an amount of time it takes caregivers to respond to nurse call requests and that keeps tracks the length of time at least one caregiver is present in a patient room based on information received by the nurse call computer from the locating equipment. The electronic medical record system is associated with a patient experience module. The patient experience module receives information from the nurse call equipment and the locating equipment. The electronic medical record system summarizes the information received, calculates and displays a patient experience counter and, calculates and displays a staff load counter associated with a plurality of patients in the healthcare facility.

According to some embodiments of the third aspect, the patient experience counter and the staff load counter associated with a patient in the healthcare facility is updated periodically.

According to some embodiments of the third aspect, the patient experience counter is included in a patient experience report and the staff load counter associated with a patient is included in a staffing optimization report.

According to some embodiments of the third aspect, the patient experience report and the staffing optimization report are accessible at one or more of the following: (1) a wireless communication device carried by at least one caregiver or (2) on a status board display located in a caregiver work area or (3) on a master nurse station computer of the nurse call system.

According to a fourth aspect of the present disclosure, a method of optimizing staffing for a plurality of caregivers to care for a plurality of patients includes acquiring, using locating equipment, the location of each of the plurality of caregivers, acquiring, using nurse call equipment, call requests from patients located in patient rooms of the healthcare facility, and utilizing a patient experience module to consider the call response data to establish a patient experience score and a staff load score. The patient rooms are divided up into at least a first unit of the healthcare facility and a second unit of the healthcare facility. The nurse call equipment includes at least one nurse call computer that keeps track of an amount of time it takes caregivers to respond to each nurse call and keeps track of how long at least one caregiver is present in each patient room based on information received by the nurse call computer from the locating equipment to establish call response data by patient. When a patient experience score varies, notify the applicable caregiver of the variation of the patient care experience score to permit the caregiver to adjust staffing in real-time to improve the patient care experience score.

According to some embodiments of the fourth aspect, the patient experience module increases the staff load counter for a patient if total number of patient calls for the patient is greater than an average benchmark for patient calls.

According to some embodiments of the fourth aspect, the patient experience module increases the staff load counter for a patient if total number of equipment calls for the patient is greater than an average benchmark for equipment calls.

According to some embodiments of the fourth aspect, the patient experience module increases the staff load counter for a patient if total number of unpredictable calls for the patient is greater than an average benchmark for unpredictable calls.

According to some embodiments of the fourth aspect, the patient experience module increases the staff load counter for a patient if the total patient visit length is greater than an average benchmark length for patient visit.

According to some embodiments of the fourth aspect, the patient experience module increases the staff load counter for a patient if the total number of patient visits is greater than an average number of patient visits.

According to some embodiments of the fourth aspect, the patient experience module divides patients into groups, and assigns a new unassigned patient to a group with the lowest total staff load counter value.

According to some embodiments of the fourth aspect, the patient experience module creates the groups by computing a descending worst fit algorithm, ranks all patients with a highest staff load counter to a lowest staff load counter, and further divides the patients into groups passed on an input parameter.

According to some embodiments of the fourth aspect, the patient experience module assigns the new patient to the group that minimizes bedside shift handoffs if there is a tie for the group with the lowest total staff load score.

According to some embodiments of the fourth aspect, the information regarding types of nurse calls is used to calculate the staff load counter.

According to some embodiments of the fourth aspect, the types of nurse calls comprises patient calls, equipment calls, and unpredictable calls.

According to some embodiments of the fourth aspect, the patient experience module provides a notification comprising staff assignments as at least one of the following: (1) a message appearing on a wireless communication device carried by at least one caregiver or (2) a message appearing on a status board display located in a caregiver work area or (3) a message appearing on a master nurse station computer of the nurse call system.

Additional features, which alone or in combination with any other feature(s), such as those listed above and those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a block diagram of a healthcare information system for use in a healthcare facility having patient rooms in various units of the healthcare facility; the healthcare information system including various servers, nurse call equipment, and locating equipment; the nurse call equipment including a nurse call server, a master nurse station coupled to the nurse call server, room stations located in the patient rooms, indicator assemblies located outside the patient rooms and coupled to the room stations via an input/output board, and nurse call buttons on a patient bed and/or on a handheld pillow speaker unit; and the locating equipment including locating tags worn by caregivers, real-time locating system receivers located in the patient rooms, and a server coupled to the receivers;

FIG. 2 is a flow chart illustrating a process for determining and periodically updating a patient experience counter while the patient is still in the hospital;

FIG. 3 is a screen shot of a graphical user interface illustrating an average patient experience counter over a period of 7 days;

FIG. 4 is a screen shot of a graphical user interface illustrating an average patient experience counter over a period of 3 days;

FIG. 5 is a screenshot of the patient experience report illustrating an average response time of different types of patient calls;

FIG. 6 is a screenshot of the patient experience report including various patient insights on a per-patient basis;

FIG. 7 is a flow chart illustrating a process for determining and periodically updating a staff load counter;

FIG. 8 is a screenshot of a staffing optimization report illustrating staff activity and number of patients assigned per staff;

FIG. 9 is a screenshot of an interface that is used to generate such staffing optimization report;

FIG. 10 is a flow chart illustrating a process for assigning new patients into groups in the healthcare facility;

FIG. 11 is a screenshot of patient grouping as evaluated by the algorithm outlined in FIG. 10; and

FIG. 12 is a screen shot of the total rounding data included in the staffing optimization report.

### DETAILED DESCRIPTION

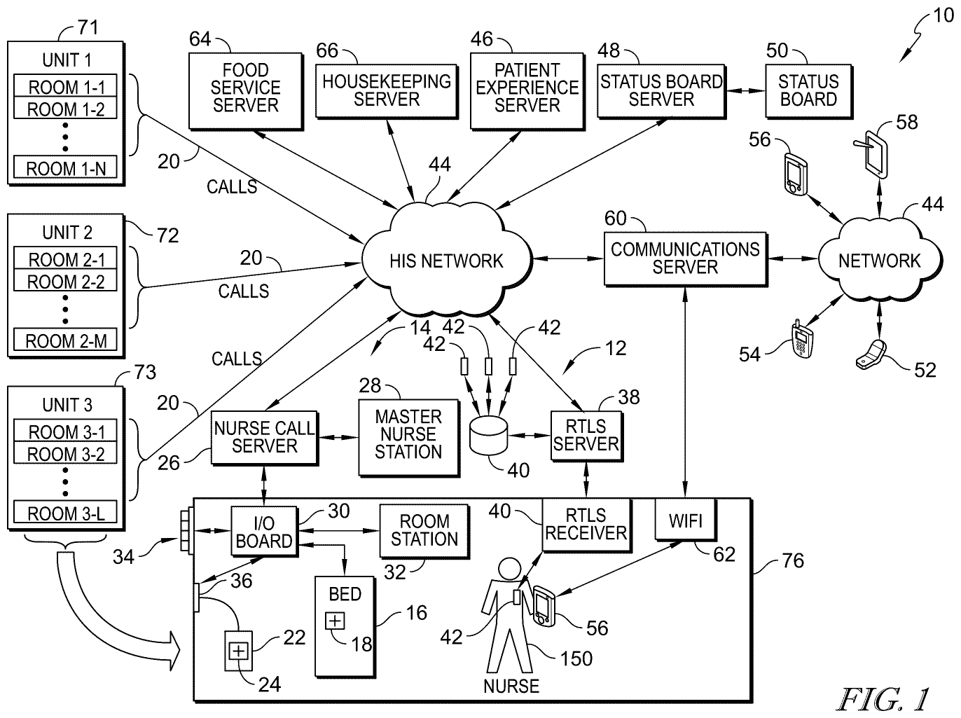

According to the present disclosure, a system 10 includes locating equipment 12 used to track the whereabouts of caregivers in a healthcare facility and nurse call equipment 14 to receive nurse call requests from patients located in patient rooms of the healthcare facility as shown diagrammatically in FIG. 1. The healthcare facility is divided into units shown diagrammatically in FIG. 1 into units 71, 72, and 73 that correspond to Unit 1, Unit 2, and Unit 3 respectively. The units 71, 72, 73 each have patient rooms which, in the illustrative example, are shown diagrammed as rooms 1-1 through 1-N for unit 1, rooms 2-1 through 2-M for unit 2, and rooms 3-1 through 3-L for unit 3. Letters N, M and L represent integers corresponding to the overall number of rooms in the corresponding unit 71, 72, or 73 and the use of different letters is intended to indicate that different units may not necessarily have the same number of rooms. While the illustrative embodiment the three units 71, 72, and 73, it should be understood that the present disclosure could apply to a facility with any number of units.

While healthcare facilities may have any number of units with various unit names and all such facilities are intended to be within the scope of the present disclosure, examples of units of a healthcare facility may include, for example, a maternity unit, a pediatrics unit, an intensive care unit, and a medical/surgical unit, just to name a few. Each room of each unit has a patient support apparatus, such as a hospital bed 16 as shown in FIG. 1 in the enlarged diagrammatic view of one patient Room N-N 76, which is intended to be illustrative of a general patient room. In illustrative embodiment, bed 16 has a nurse call button 18, which is pressed by a patient send a nurse call to the nurse call equipment 14 of system 10. The disclosed room also has a handheld pillow speaker unit 22 with its own nurse call button 24 as shown diagrammatically in FIG. 1. Nurse calls originating from the various patient rooms of units 1-3 in response to buttons 18, 24 being pressed are represented by arrows 20 in FIG. 1. Buttons 18, 24 and/or handheld pillow speaker unit 22 may be considered part of nurse call equipment 14 in some embodiments. One or more may be

The nurse call equipment 14 includes a nurse call server 26 and a master nurse station 28 as shown diagrammatically in FIG. 1. It should be appreciated that some healthcare facilities may have two or more servers 26 and two or more master nurse station 28. For example, a master nurse station 28 may be provided for each unit of the healthcare facility if desired. In the illustrative example, the nurse call equipment 14 also includes, in each room 76, an input/output (I/O) board 30 that receives nurse call signals from the one or more beds 16 and units 22 in the room. The nurse call equipment 14 also includes at least one room station 32 in each room 76 and at least one indicator assembly 34 (sometimes referred to as a dome light assembly or just a dome light) located in a hallway adjacent to the room. Each I/O board 30 is communicatively coupled to a respective server 26, room station 32, and indicator assembly 34. In the illustrative example, a wall connector 36 is provided for coupling to a cable 78 extending from unit 22 and the connector 36 is coupled to I/O board 30.

I/O board 30 passes any nurse calls 20 made by a patient using button 18 or button 24 to server 26. Information concerning the nurse calls 20 made in the various rooms is displayed on display screens, such as graphical user interfaces (GUI's), of room stations 32 and master nurse station 28. In some embodiments, additional components are included in nurse call equipment 14 such as, for example, bed interface units (BIU's), routers, gateways, cabling, etc. One example of nurse call equipment 14 contemplated by the present disclosure is the NAVICARE® nurse call system available from Hill-Rom Company, Inc. Additional details of suitable nurse call equipment 14 that may be included in system 10 are shown and described in U.S. Pat. Nos. 8,598,995; 8,384,526; 8,169,304; 8,046,625 and 7,319,386; each of which is hereby incorporated by reference herein for the disclosure of nurse call systems to the extent not inconsistent with the present disclosure, which shall control as to any inconsistencies.

Still referring to FIG. 1, locating equipment 12 includes a real-time locating system (RTLS) server 38, a multitude of RTLS receivers 40 located throughout the healthcare facility, and a multitude of RTLS badges tags 42 that are worn by caregivers such as a nurse 150, such as by being clipped to the clothing of the caregiver 150 for example. As the caregiver 150 travel throughout the healthcare facility, tags 42 transmit signals to the receivers 40 at the caregiver's location. The signals from tags 42 each include a unique tag identification code or number (ID) that is correlated in a database of server 38 to the identity of the caregiver to which the particular tag 42 has been assigned. Receivers 40 each transmit to server 38 a receiver ID along with the tag ID of any tags 42 within the reception range of the particular receiver 40. The receiver ID is correlated in the database of server 38 to a particular location, such as a room or hallway, of the healthcare facility. Thus, based on the transmissions from the various receivers 40 of receiver ID and tag ID, the server 38 determines the location of each of the caregivers having an associated tag 42 within the healthcare facility. The tags 42 may be attached to particular pieces of equipment (e.g., beds, wheelchairs, IV pumps, vital signs monitors, etc.) so that the locating equipment 12 is able to track the locations of the equipment in a similar manner, if desired.

It is within the scope of the present disclosure for the locating equipment 12 to implement any of a variety of wireless communication technologies to achieve the function of tracking the whereabouts of caregivers in a healthcare facility. Radio frequency (RF) including WiFi (i.e., 802.11) or Bluetooth (BT), infrared (IR), ultrasonic (US), ultra wide band (UWB), and so forth are a few examples of such technologies. In some embodiments, for example, locating units (not shown) mounted throughout the healthcare facility transmit IR signals to tags 42. The IR signals are encoded with a location ID, which correlates in the database of the server 38 to the location of the locating unit. The tags 42 then transmit the location IDs and tag IDs as an RF signal the receivers 40. Thus, the tags 42 in such embodiments have an IR receiver and an RF transmitter. The receivers 40 transmit the location ID's (sent originally via IR) and the tag ID's to the server 38 for correlation. Thus, the receivers 40 may be more accurately characterized as receiver/transmitters or transceivers in some embodiments since they both transmit and receive. Such a system that uses IR and RF technology for locating is marketed, for example, by Centrak Inc. of Newtown, Pennsylvania.

The nurse call equipment 14 and locating equipment 12 are communicatively coupled to one another by a healthcare information system (HIS) network 44, which is illustrated diagrammatically in FIG. 1, but which is intended to represent all of the infrastructure of a healthcare facility, such as gateways, routers, cabling, network servers, and the like, used to interconnect computer devices including various servers, such as the servers 26, 38, in a healthcare facility. In this regard, the nurse call server 26 of the nurse call equipment 14 receives information from the RTLS server 38 of the locating equipment 12 regarding the location of various caregivers having tags 42, as they travel throughout the healthcare facility.

According to this disclosure, each time a patient places a nurse call 20; the nurse call server 26 keeps track of the amount of time that elapses subsequent to the respective nurse call 20 being placed. Thus, each nurse call 20 has a timer associated therewith, which is implemented in nurse call software executed by the server 26. When a caregiver wearing one of the tags 42 enters a particular room having an outstanding nurse call 20, the nurse call server 26 stops the timer of the pending nurse call 20 for that particular room, either substantially in real time upon entry of the caregiver into the room or after a threshold amount of the caregiver's presence in the room, such as one to five minutes, for example. As such, the nurse call server 26 is able to keep track of nurse call response times for each nurse call 20. The server 26 also has data concerning the overall total number of nurse calls from each patient room at any given time. The server 26 also has data concerning the completion status of each patient round undertaken by the caregiver, i.e., if the rounds were completed, missed, partial, or on hold (“rounding data 326”). Additionally, when a caregiver wearing one of the tags 42 enters a particular room having a pending nurse call 20, the amount of time the caregiver spends in the patient room is tracked. Data from the server 38 also indicates how long each caregiver having a respective tag 42 is present in each patient room.

According to this disclosure, a patient experience server 46 is coupled to the network 44. The patient experience server 46 includes software, sometimes referred to herein as a patient experience module 46, which monitors and analyzes the data associated with the servers 26, 38. In some embodiments, the patient experience module 46 is included in server 26 and/or server 38 in which patient experience server 46 may be omitted, if desired. Thus, the patient experience module 46 receives data concerning call volume (i.e., number of nurse calls 20 at any given time), nurse call timer information at any given time, and response times associated with each of the nurse calls 20 that are placed. In some embodiments, the patient experience module 46 receives information from the nurse call equipment 14 regarding different types of nurse calls 20 that are placed by the patients. The types of nurse calls 20 may include, for example, pain calls, bathroom calls, bed exit calls, and normal calls (collectively “call data 334”).

The patient experience module 46 also receives information concerning staffing assignments 330 including nurse/caregiver staffing assignments, patient data 340, nurse and/or caregiver location data 142, and rounding data 326. The server 26 also provides to the patient experience module 46 information concerning which unit (e.g., units 1-3) each of the calls originated. The patient experience module 46 prepares a patient experience report and a staff optimization report comprising a patient experience counter for each patient based on all of the information received.

The current disclosure is directed to the use of an electronic medical record plus (“EMRP”) system that can generate and summarize data periodically in real time. Evaluation of real time data results in more frequent and up-to-date patient experience assessment. This is useful for shift leaders who can subscribe to receive such periodic assessments. Such real time evaluation also allows for an analysis of patient experiences for more details including staff visits, rounding compliance, and patient calls.

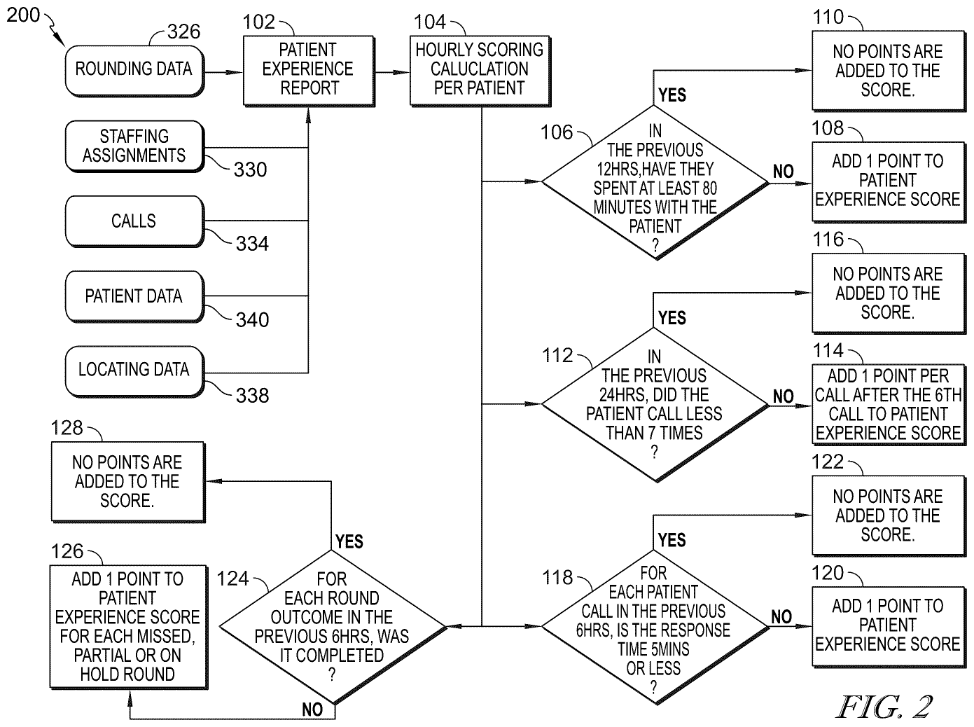

Referring now to FIG. 2, a flow chart 200 illustrates a process for updating the patient experience counter. The patient experience counter is illustrative of the functionality of the patient experience module 46 associated with the EMRP system that can generate and summarize data periodically. The patient experience module 46 and the EMRP system are embodied as software stored in memory of one or more of the servers 26, 38, 46 and executed by the respective processors of the servers 26, 38, 46. The patient experience report is prepared based on rounding data 326, staffing assignment data 330, nurse call data 334, patient data 340, and location data as shown in step 102. The patient experience module 46 prepares the patient experience report on an hourly basis as shown in step 104. All patient experience reports are stored on the server 26.

The patient experience module 46 determines an amount of time that a caregiver spends in a patient's room during a response to a nurse call 20. If the caregiver spends a sufficient amount of time in the patient's room (e.g., more than a threshold amount), then the patient experience module 46 updates a rounding time for the particular patient or sends a message to one or more of the servers 26, 38, 48 to update the rounding timer. In step 106, the patient experience module 46 determines if the nurse has spent at least a threshold amount of time with a particular patient in the previous 12-hour period. If the nurse has not spent at least the threshold amount of time with a particular patient in the previous 12-hour period, the patient experience module 46 adds one point to the patient experience counter for that particular patient in step 108. If the nurse has spent at least the threshold amount of time with a particular patient in the previous 12-hour period, no points are added to the patient experience counter for that particular patient in step 110. The threshold amount of time may be determined by the nurse or caregiver. In some embodiments, the threshold amount of time may be about 80 minutes. As will be described below, the points in the patient experience counter are used to assess and adjust staffing to tailor the patient care delivered for a particular patient.

The patient experience module 46 receives nurse calls 20 and tracks the overall number of calls received. The patient experience module 46 may segregate the nurse calls 20 into the various call types. The patient experience module 46 may also segregate the nurse calls 20 by unit type. In step 112, the patient experience module 46 determines if the patient has called any nurse or caregiver less than seven times in the previous 24-hour period. If the patient has not called any nurse or caregiver less than seven times in the previous 24-hour period, the patient experience module 46 adds one point to the patient experience counter for that particular patient in step 114. If the patient has called any nurse or caregiver less than seven times in the previous 24-hour period, no points are added to the patient experience counter for that particular patient in step 116.

The patient experience module 46 determines average response times when responding to nurse calls 20. Averages can be taken at time intervals that are selected by a user of the patient experience module 46. For example, the patient experience module 46 can be programmed to calculate the averages hourly, by shift, daily, etc. As caregivers respond to the various nurse calls 20 in the various units, the patient experience module 46 determines the response time for each call (i.e., how long it took before a caregiver attended to the nurse call 20). In step 118, the patient experience module 46 determines if the nurse response time for every patient call in the previous 6-hour period was five minutes or less. If the patient experience module 46 determines that the nurse response time for every patient call in the previous 6-hour period was not five minutes or less, the patient experience module 46 adds one point to the patient experience counter for that particular patient in step 120. If the patient experience module 46 determines that the nurse response time for every patient call in the previous 6-hour period was five minutes or less, no points are added to the patient experience counter for that particular patient in step 122.

In step 124, the patient experience module 46 evaluates each round outcome for a particular patient in the previous 6-hour period. If each round undertaken in the previous 6-hour period was completed, no points are added to the patient experience counter for that particular patient in step 126. The patient experience module 46 adds one point to the patient experience counter for every missed, partial, or on-hold round in step 128.

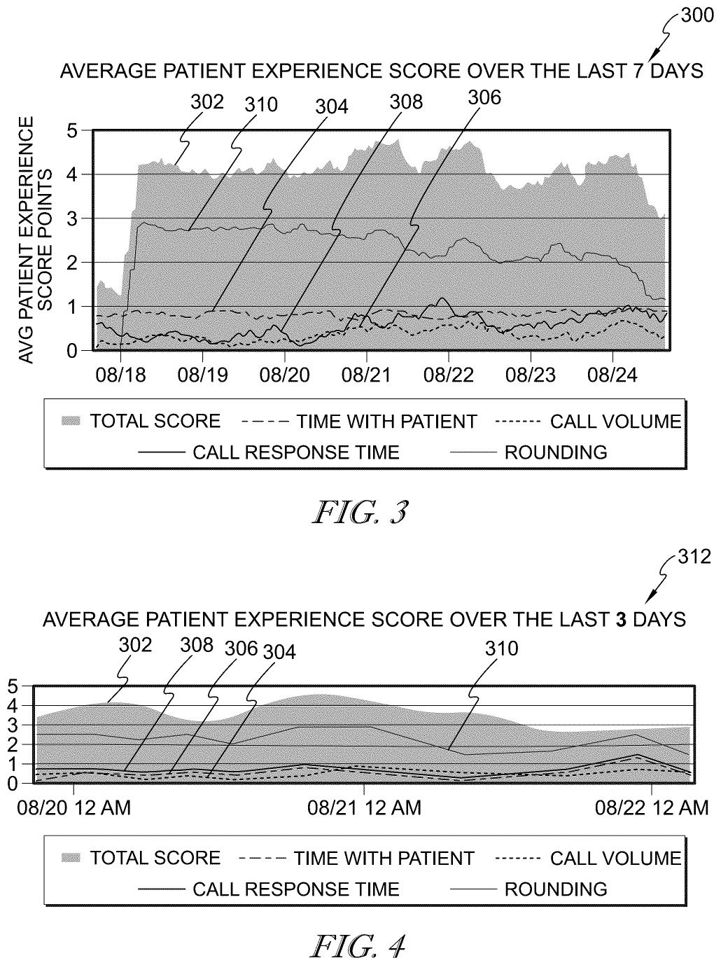

The patient experience module 46 evaluates and updates the patient experience counter for each patient periodically (e.g., hourly). The patient experience module 46 summarizes the data from the locating equipment 12 and nurse call equipment 14 to develop notifications including progress reports and GUIs. The patient experience module 46 stores the patient experience counter as determined for patients over their stay in the hospital. The patient experience module 46 is configured to store, evaluate, and notify an average patient experience counter over a time-period as shown displayed on a a GUI in FIGS. 3-4. An example screenshot 300 of the presentation of the average patient experience counter for a 7 day period is shown in FIG. 3 and a screenshot 312 of the average patient experience counter for a 3 day period is shown in FIG. 4. The screenshots 300 and 310 are examples of data presented on the GUI of master nurse station 28 as shown in FIG. 1. In the illustrative embodiment, the screenshot displays a total patient experience score 302, a measure of the time spent with the patient 304, a measure of call volume 306, a measure of call response time 308, a measure of the rounding 310. These values are normalized scores on the chart to show the total points for the patient experience 302. The total rounding data may include real-time rounding information concerning all patients including, the percentage of rounds on hold, the percentage of rounds missed, the percentage of rounds completed.

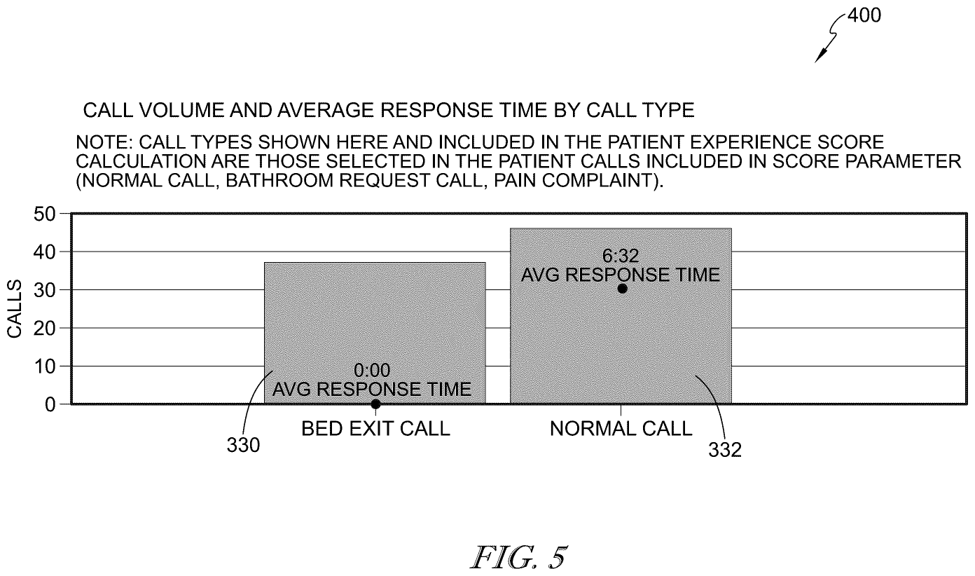

As shown in FIG. 5, the patient experience module 46 determines on a per-patient basis, the average response times based on patient call type. For example, as shown in a GUI screenshot 400, the average response time for all thirty-eight of the bed exit calls 330 for a particular patient was 0:00, indicating that the call were responded to immediately. The average response time for all forty-six of the normal calls 332 made by the same patient was about 6 minutes and 32 seconds. The patient experience module 46 is operable to include this information in the patient experience report and overall score.

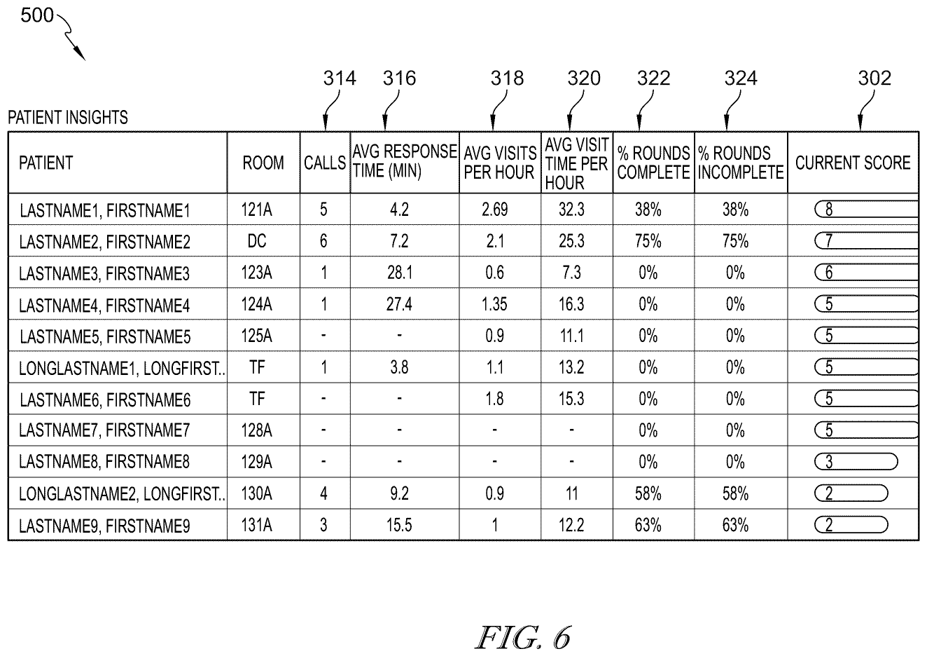

Additional information summarized by patient is shown in a screenshot 500 in FIG. 6. The patient experience module 46 determines various patient insights on a per-patient basis culminating in a current patient experience score 302. The patient insights may include the number of calls 314, average response times 316, average caregiver/nurse visits per hour 318, the average time that caregivers spent in each of the patient rooms 320, and the percentage of rounds that are complete 322 and incomplete 324. The averages are typically taken hourly, but can be taken at time intervals that are selected by a user of the patient experience module 46. The patient experience counter is updated based on these hourly calculations. Alternatively, the patient experience module 46 can be programmed to calculate the averages hourly, by shift, daily, etc., updating the patient experience score 302 accordingly. The patient experience report includes the information shown in FIG. 6, and is updated hourly. The patient experience report is accessed at the master nurse station 28 or by caregivers and nurses at their stations.

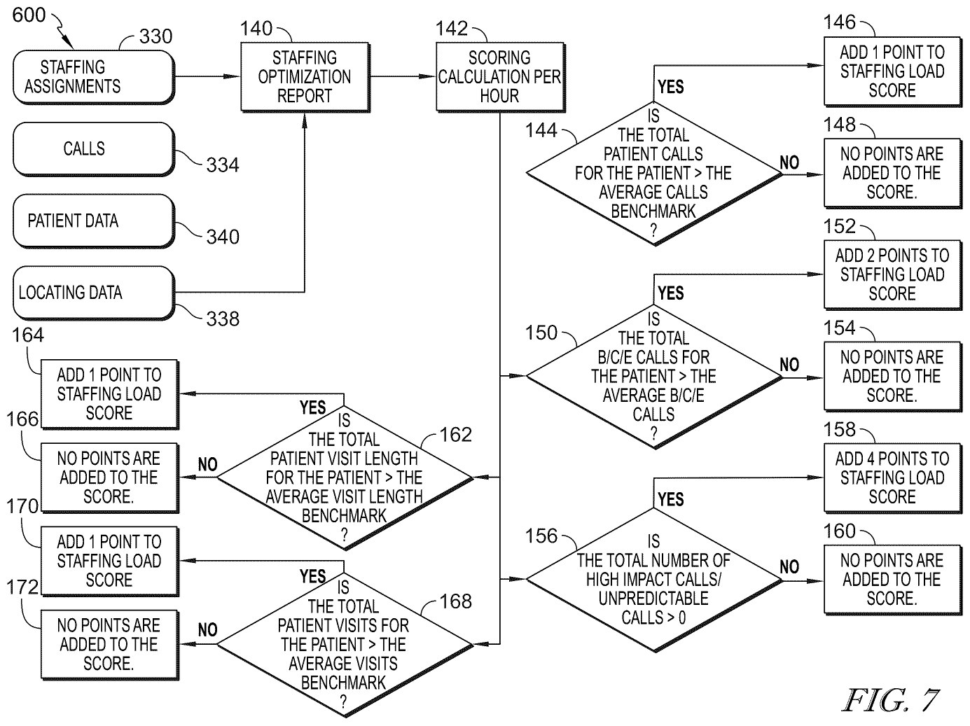

One approach to the analysis of staffing levels and patient experience in a healthcare facility is shown in an algorithm 600 in FIG. 7. Staffing depends on the number and frequency of patient calls, B/C/E calls, and other unpredictable calls for the patients. The method of optimizing staffing includes collecting real time operational data, including clinical data and data regarding staff on a particular unit of a healthcare facility and maintaining a staff load counter score 380 for each patient. A flow chart 600 is provided for determining the staff load counter 380 and is illustrative of the functionality of the patient experience module 46 which, as mentioned above, is embodied as software stored in memory of one or more of servers 26, 38, 46 and executed by the respective processors of the servers 26, 38, 46. The patient experience module 46 may be associated with the electronic medical record plus system. The staff optimization report is prepared in step 340 based on staffing assignments 350, nurse call data 334, patient data 340, and location data 616. The patient experience module 46 prepares the staff optimization report based on the staff load counter 380 calculated for each patient as shown in step 142. All staff optimization reports are stored in the server 26. The staff load counter 380 for each patient is calculated as shown in steps 144, 150, 156, 162, and 168.

In step 144, the patient experience module 46 determines if the total number of patient calls for a particular patient is greater than an average benchmark previously determined. If the total number of patient calls for a particular patient is greater than an average benchmark previously determined, the patient experience module 46 adds one point the staff load counter 380 for that particular patient in step 146. If the total number of patient calls for a particular patient is not greater than an average benchmark previously determined, no points are added to the staff load counter 380 for that particular patient in step 148.

In step 150, the patient experience module 46 determines if the total B/C/E calls for a particular patient is greater than an average bed, chair, equipment calls for a patient as previously determined. If the total B/C/E calls for a particular patient is greater than an average B/C/E calls for a patient as previously determined, the patient experience module 46 adds two points to the staff load counter 380 for that particular patient in step 152. If the total B/C/E calls for a particular patient is not greater than an average B/C/E calls for a patient as previously determined, no points are added to the staff load counter 380 for that particular patient in step 154.

In step 156, the patient experience module 46 determines if the total number of unpredictable calls for a particular patient is greater than zero. If the total number of unpredictable calls for a particular patient is greater than zero, the patient experience module 46 adds two points to the staff load counter 380 for that particular patient in step 158. If the total number of unpredictable calls for a particular patient is not greater than zero, no points are added to the staff load counter 380 for that particular patient in step 160.

In step 162, the patient experience module 46 determines if the total patient visit length for a particular patient is greater than an average length benchmark previously determined. If the total patient visit length for a particular patient is greater than an average length benchmark previously determined, the experience module 46 adds one point to the staff load counter 380 for that particular patient in step 164. If the total patient visit length for a particular patient is not greater than an average length benchmark previously determined, no points are added to the staff load counter 380 for that particular patient in step 166.

In step 168, the patient experience module 46 determines if the total number of patient visits for a particular patient is greater than an average benchmark number previously determined. If the total number of patient visits for a particular patient is greater than an average benchmark number previously determined, the patient experience module 46 adds one point to the staff load counter 380 for that particular patient in step 170. If the total number of patient visits for a particular patient is not greater than an average benchmark number previously determined, no points are added to the staff load counter 380 for that particular patient in step 172.

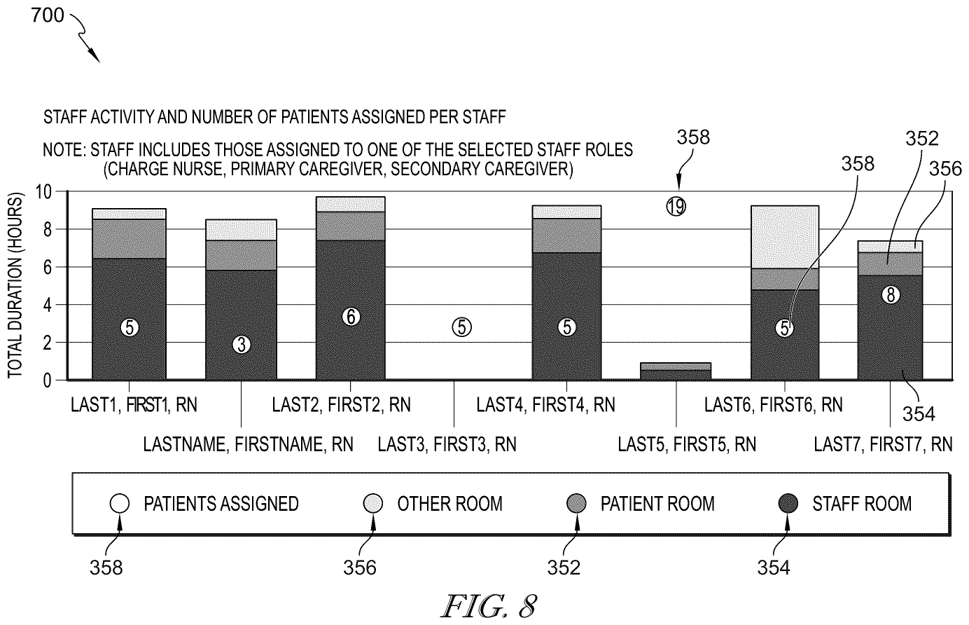

The patient experience module 46 is operable to include information illustrated in FIG. 8 in the staffing optimization report. As shown in a GUI screenshot 700, the patient experience module 46 is operable to determine and display where each nurse or caregiver 150 spends their time during their shift or for every 24-hour period. For example, the bar charts for location data for the caregivers 150 show, for each caregiver, the time spent in a patient room 352, staff room 354, or other room 356. Additionally, the patient experience module 46 is operable to determine the number of patients assigned to each nurse or caregiver 150 and provide that on the bar charts as a bubble 358 for each caregiver 150.

In other embodiments, other information about the patient can be considered in developing a load score. Examples of other patient factors to be considered include:
fall risk factor for the patient
patient weight (considered for patient handling)
a do not resuscitate directive for the patient
patient acuity
patient incontinence
procedures and therapies outlined in a care plan (e.g. dialysis, ventilation, CPAP, breathing treatments, presence of central or PICC lines)
levels of co-morbidity
wound care protocols for the patient
presence of drains
medication frequency
feeding tubes
infection precautions for the patient
behavioral/mental health diagnoses
emotional needs
familial support
needs for translator support/language barriers

Similarly, caregiver capabilities and experience may also be considered in developing a load score or staffing optimization. Examples of caregiver factors to be considered include:
caregiver certifications
caregiver education
known efficiency
years of experience
licensure
speed
status (float, travel, PRN)
skills

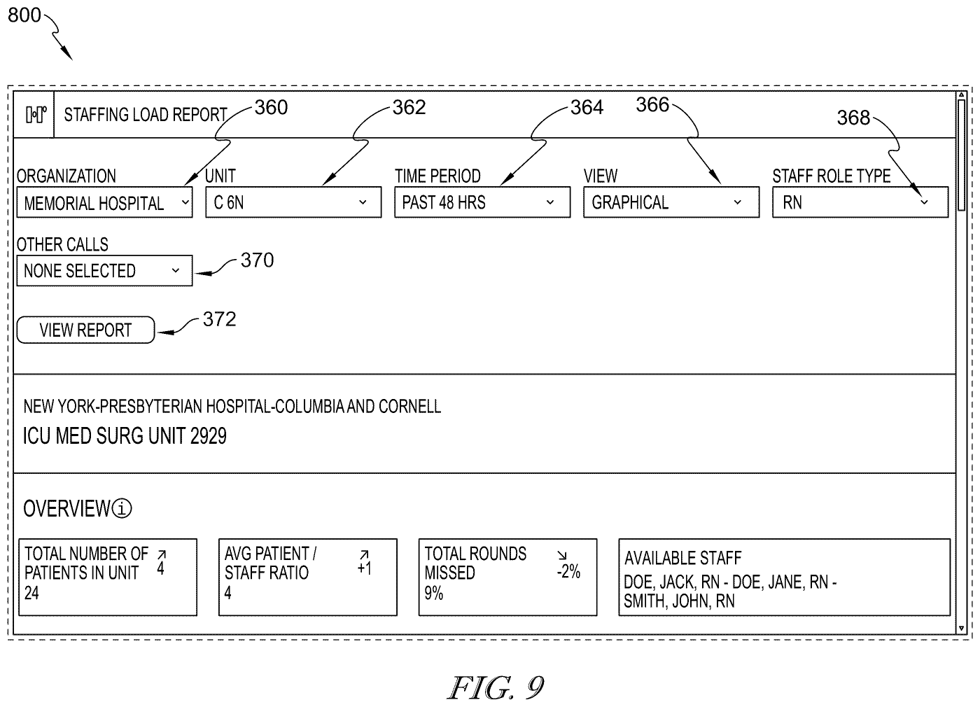

A staffing optimization report that include staff load scores 380 can be generated by a nurse or caregiver for any specified time-period. As an example, FIG. 9 is GUI screenshot of an interface 800 that may be used to generate such reports. The staffing optimization can be updated hourly. The staffing optimization report relieves stress on caregivers by exploring staff workload. The staffing optimization report is not based on acuity, unlike traditional burden calculators. The staffing optimization report objectively keeps track of the staff load counter, highlighting staff burden and suggesting staff available for future assignments. The staffing optimization report can be accessed at the master nurse station 28 or by caregivers and nurses at their stations. Alternatively, the staffing optimization report may be projected continuously and updated periodically at the master nurse station 28. As an example, a staffing load report similar to that shown in FIG. 11 can be created by choosing several drop down selections. For example, the specific organization can be selected at drop down 360. A particular unit of that organization may be selected at drop down 362 to further refine the data. The time period for the load is selected at drop down 364 and the type of view is selected at drop down 366. The staffing optimization report can be represented via text, a table or a graph by selecting the appropriate value at drop down 366. The staffing optimization report can include different groups of caregivers by selecting the button 368. The types of calls to be included are selected at drop down 370 and the report is initiated by selecting the button 372.

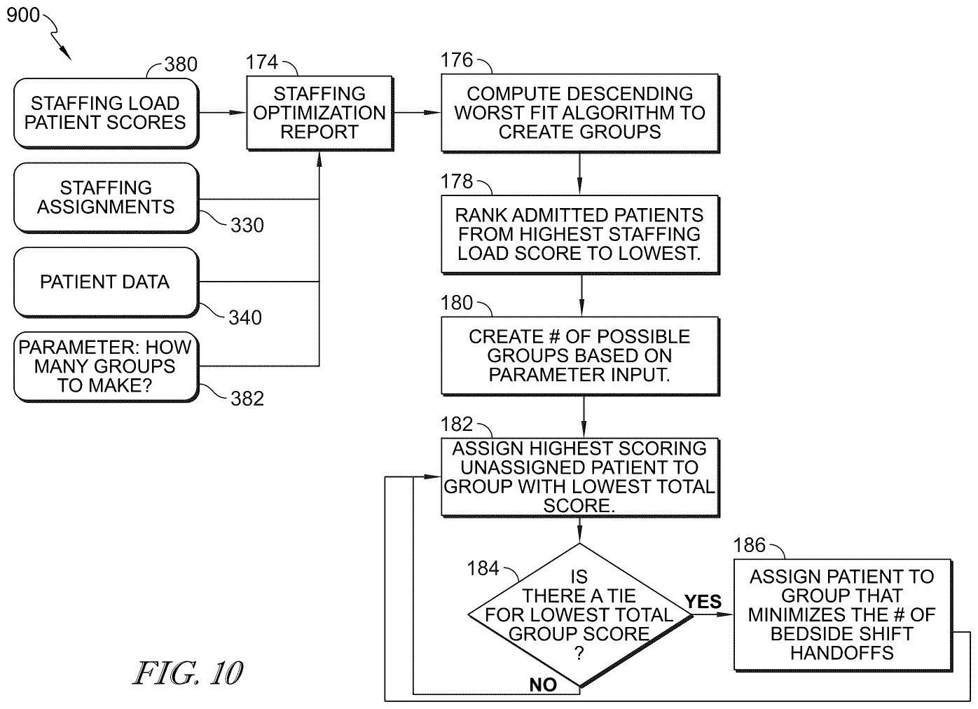

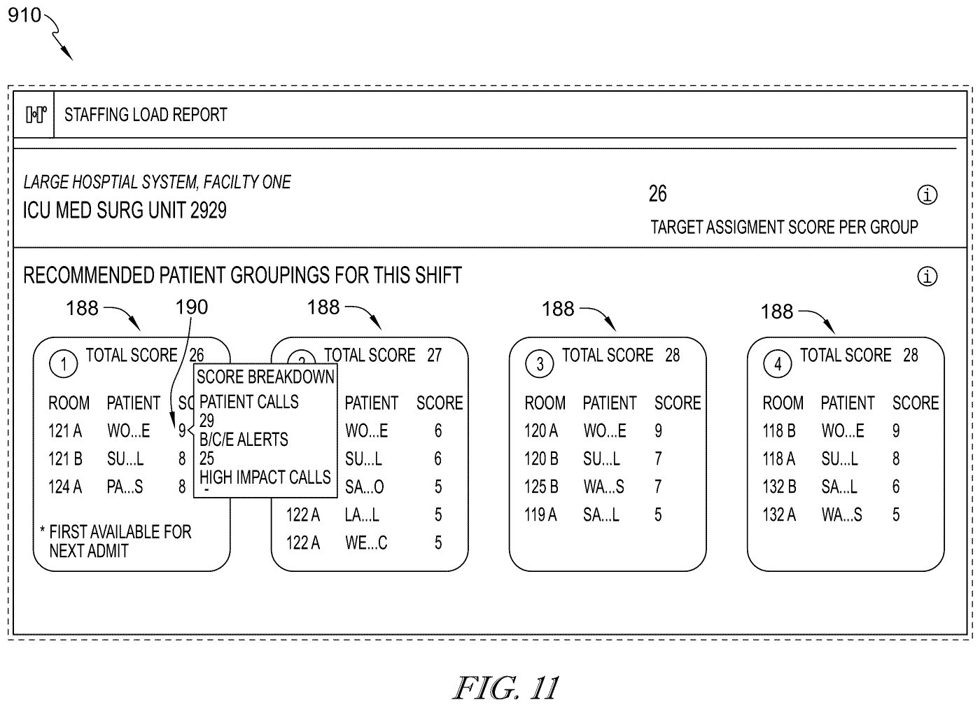

Patients are assigned to groups based on the staffing optimization report. One method of assigning new patients into groups in a healthcare facility is shown by a flowchart 900 in FIG. 10. Based on the staffing optimization report generated in step 174, the patient experience module 46 is operable compute a descending worst fit algorithm to create patient groups in step 176. The staffing optimization report is based on the staffing assignments 330, patient data 340, staff load counter 380 for each patient as determined previously and any parameter input about number of patient groups at 382. The patient experience module 46 is operable to rank all the admitted patients in the healthcare facility from highest to the lowest based on the current value of the staff load counter 380 for each patient in step 178. The patient experience module 46 is operable to create number of possible groups based on the parameter input in step 180. The patient experience module 46 is operable to assign the new, unassigned patient to the group with the lowest total score in step 182. Once the new patient is assigned to a group, the patient experience module 46 is operable to determine if there is a tie for the lowest group based on the new assignment in step 184. If there are no ties, the patient experience module 46 is operable to assign the next unassigned patient to the group with the lowest total score in step 182. If there is a tie in step 184, the patient experience module 46 is operable to assign the new patient to the group that minimized the number of bed-shift handoffs amongst the tied groups in step 186.

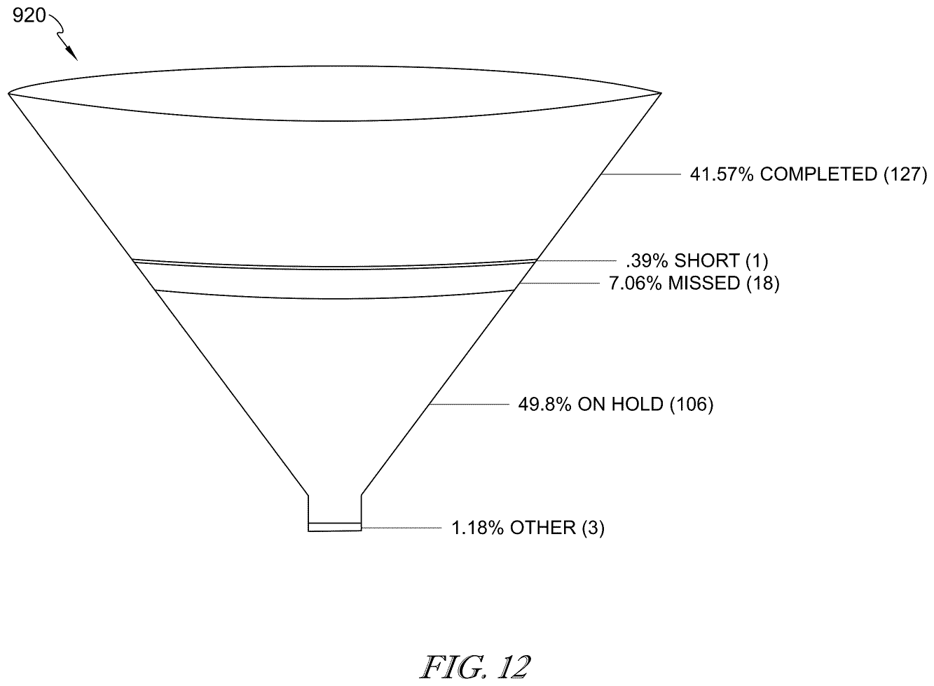

FIG. 11 illustrates an example patient grouping as evaluated by the algorithm outlined in FIG. 10. A GUI screen shot 910 of the staffing optimization report, which can be accessed by the caregiver or user illustrates a plurality of patient groupings 188, the patients comprised in each grouping 188, and the staff load counter 380 associated with each patient. By selecting the staff load score 380 associated with a particular patient, the user can access an accounting of different calls associated with that patient. In some embodiments, such as that shown in FIG. 12, the staffing optimization report may include total rounding data 144 presented on a screenshot 920 which displays the rounding data in a graphical format that provides a quick reference for the status of the rounding data. The total rounding data 144 includes data that including the percentage of completed, short, missed, and on-hold rounds. While the data is presented as a funnel graph in FIG. 12, it should be understood that any of a number of graphical representations may be used to provide visualization of the rounding data, including pie charts, exploded pie charts, bar charts, heat maps, or the like.

In some scenarios contemplated by this disclosure, patients have their own tablets or smart phones on which communication software is installed for interfacing with system 10 via network 44. Alternatively or additionally, a healthcare facility may provide the patient with a tablet or other digital device for use during their stay at the facility. Various call type icons are displayed on the GUI of the patient's personal digital device in connection with the communication software. Selection of a call type icon on the patient's GUI results in a nurse call 20 being sent to server 26 with information concerning the call type. In this regard, see, for example, U.S. Patent Application Publication No. 2016/0055299, which is hereby incorporated by reference herein to the extent not inconsistent with the present disclosure, which shall control as to any inconsistencies. Beds 16 with patient GUI's for placing specific call types are also within the scope of this disclosure. See, for example, U.S. Pat. No. 9,286,441, which is hereby incorporated by reference herein to the extent not inconsistent with the present disclosure, which shall control as to any inconsistencies.

It is also contemplated by this disclosure that a variety of different types of alerts and notifications can be initiated by the patient experience module 46. For example, when a nurse call 20 is placed by a patient in a particular room, one of the lights of indicator assembly 34 associated with the room becomes illuminated in a first state, such as for example, a non-flashing amber or yellow light. In some embodiments, if the nurse call 20 is not answered by a caregiver visiting the patient room within a first threshold amount of time, the patient experience module 46 signals the particular indicator assembly 34 via server 26 and I/O board 30 to cause the yellow light to flash on and off. If the nurse call 20 is not answered by a caregiver visiting the patient room within a second threshold amount of time, the patient experience module 46 signals the indicator assembly 34 via server 26 and I/O board 30 to illuminate a red light and to turn off the flashing yellow light. In some embodiments, if a caregiver at master nurse station 28 opens up a communication channel to speak with the patient placing the nurse call, then server 26 signals indicator assembly 34 via I/O board 30 to illuminate a green light. Thus, the lights of indicator assemblies 34 serve as one type of alert or notification to caregivers as to the status of the associated nurse calls 20 and whether nurse calls 20 are being answered by caregivers with visits to the patient rooms within acceptable time thresholds.

Alerts and notifications initiated by the patient experience module 46 include messages on display screens of various computer devices in some embodiments. For example, as shown in FIGS. 3-4, the patient experience module 46 may send a signal to server 26 to cause a message or icon to be displayed on the GUI of master nurse station 28 to indicate an average value for the patient experience counter over the previous 7 days or 3 days. The message or icon to be displayed on the GUI of master nurse station 28 may also be accessed by the caregivers 150 on their personal devices. The patient experience module 46 is operable to determine the average value of the patient experience counter over any time-period. In some embodiments, system 10 includes a status board server 48, which communicates with servers 26, 38, 46 via network 44. One or more status boards 50 are coupled to server 48. The patient experience module 46 may periodically send a signal to server 48 to cause a message or icon to be displayed on the status board 50 in connection with one or more patients to indicate their average patient experience counter, for example, when that patient's experience score indicates change that requires attention by the caregivers 150. Status board 50 typically includes a display screen that is much larger than a display screen of a regular computer or master nurse station 28. The status board 50 is usually mounted in an area that is highly trafficked by caregivers so as to be readily visible by caregivers during their shifts. For additional details of a suitable status board 50, see U.S. Pat. No. 8,779,924, which is hereby incorporated by reference herein to the extent not inconsistent with the present disclosure, which shall control as to any inconsistencies.

Further, according to this disclosure, computer devices 52, 54, 56, 58 that receive alerts and notifications initiated by the patient experience module 46 include wireless communication devices carried by caregivers. As shown in FIG. 1, such wireless communication devices include, for example, telephone handsets 52, pagers 54, smart phones 56, and tablet computers 58. Each of these wireless devices 52, 54, 56, 58 have a display screen on which the message and/or alert is displayed. One or more communications servers 60 are provided in system 10 in the illustrative example to facilitate the communication of such alerts or notifications from one or more of servers 26, 38, 46 to devices 52, 54, 56, 58. One or more of servers 60 may be a voice over Internet Protocol (VOIP) server in some embodiments. As shown in FIG. 1, system 10 includes other communication infrastructure, such as wireless access points 62 that are communicatively coupled to server 60 and that are mounted throughout the healthcare facility.

Although certain illustrative embodiments have been described in detail above, many embodiments, variations and modifications are possible that are still within the scope and spirit of this disclosure as described herein and as defined in the following claims.

What is claimed is:

1. A system for providing real-time patient experience feedback to a caregiver comprising
locating equipment configured to track the location of caregivers in a healthcare facility,
nurse call equipment configured to receive nurse call requests from patients located in patient rooms of the healthcare facility, the nurse call equipment including at least one nurse call computer that tracks caregiver response time for nurse call requests, how long at least one caregiver is present in a patient room based on information received by the nurse call computer from the locating equipment, how often a patient calls the caregivers, and the status of rounds that the caregivers undertake, and
a patient experience module configured to receive information from the nurse call equipment, the locating equipment, and information about staffing assignments, update a patient experience counter associated with a particular patient based on the information received on an hourly basis, and issue a notification to a caregiver of the change in patient experience counter.

2. The system of claim 1, wherein the patient experience module is configured to increase the patient experience counter for a patient if caregivers have spent at least a threshold amount of time with the patient in the previous 12 hours.

3. The system of claim 1, wherein the threshold amount of time is 80 minutes.

4. The system of claim 1, wherein the patient experience module is configured to increase the patient experience counter for a patient if the patient has called the caregivers at least a threshold amount of hours with the patient in the previous 24 hours.

5. The system of claim 1, wherein the threshold amount of hours is 7 hours.

6. The system of claim 1, wherein the patient experience module is configured to increase the patient experience counter for a patient if a caregiver response time to each patient call from the patient in the previous 6 hours has been more than a threshold response time.

7. The system of claim 1, wherein the notification further comprises at least one of the following: (1) a message appearing on a wireless communication device carried by at least one caregiver or (2) a message appearing on a status board display located in a caregiver work area, and the notification includes whether nurse calls are being answered by caregivers within acceptable time thresholds.

8. The system of claim 1, wherein the patient experience module is configured to increase the patient experience counter for a patient for every partial, missed, or on-hold round in the previous 6 hours.

9. The system of claim 8, wherein the nurse call equipment is configured to include nurse call input devices including handheld pillow speaker units having a plurality of nurse call buttons, each nurse call button corresponding to a call type that is different than each of the other nurse call buttons.

10. A system for providing real-time patient experience feedback to a caregiver comprising
locating equipment configured to track the location of caregivers in a healthcare facility,
nurse call equipment configured to receive nurse call requests from patients located in patient rooms of the healthcare facility, the nurse call equipment including at least one nurse call computer that tracks caregiver response time to each nurse call request, how long at least one caregiver is present in each patient room based on information received by the nurse call computer from the locating equipment, how often each patient calls the caregivers, and status of each round outcome that the caregivers undertake, and
a patient experience module configured to receive information from the nurse call equipment, the locating equipment, and information about staffing assignments, update a staff load counter associated with a plurality of patients based on the information, divide the plurality of patients into groups based, and assign a new unassigned patient to a group with the lowest total staff load counter value.

11. The system of claim 10, wherein the patient experience module is configured to create the groups by computing a descending worst fit algorithm, rank the plurality of patients with a highest staff load counter to a lowest staff load counter, and divide the plurality of patients into groups based on an input parameter.

12. The system of claim 10, wherein the patient experience module is configured to assign the new patient to a group that minimizes bedside shift handoffs if there is a tie for the group with the lowest total staff load score.

13. The system of claim 10, wherein the information regarding types of nurse calls is used to calculate the staff load counter.

14. A method of optimizing staffing for a plurality of caregivers to care for a plurality of patients comprising,
acquiring, using locating equipment, the location of each of the plurality of caregivers,
acquiring, using nurse call equipment, call requests from patients located in patient rooms of the healthcare facility, the patient rooms being divided up into at least a first unit of the healthcare facility and a second unit of the healthcare facility, the nurse call equipment including at least one nurse call computer that keeps track of an amount of time it takes caregivers to respond to each nurse call and that keeps track of how long at least one caregiver is present in each patient room based on information received by the nurse call computer from the locating equipment to establish call response data by patient,
utilizing a patient experience module to consider the call response data to establish a patient care experience score and a staff load score, and
when a patient experience score varies, notify the applicable caregiver of the variation of the patient care experience score to permit the caregiver to adjust staffing in real-time to improve the patient care experience score.

15. The method of claim 14, wherein the patient experience module increases the staff load counter for a patient if total number of patient calls for the patient is greater than an average benchmark for patient calls.

16. The method of claim 14, wherein the patient experience module increases the staff load counter for a patient if total number of equipment calls for the patient is greater than an average benchmark for equipment calls.

17. The method of claim 14, wherein the patient experience module increases the staff load counter for a patient if total number of unpredictable calls for the patient is greater than an average benchmark for unpredictable calls.

18. The method of claim 14, wherein the patient experience module increases the staff load counter for a patient if the total patient visit length is greater than an average benchmark length for patient visit.

19. The method of claim 14, wherein the patient experience module increases the staff load counter for a patient if the total number of patient visits is greater than an average number of patient visits.

20. The method of claim 14, wherein the patient experience module divides patients into groups, and assigns a new unassigned patient to a group with the lowest total staff load counter value.
